(19) United States
(12) Patent Application Publication
(10) Pub. No.: US 2006/0253055 A1
(43) Pub. Date: Nov. 9, 2006

(54) Cervical collar cover

(76) Inventor: Kristyl R. Lindbery, Ventura, CA (US)

Correspondence Address: English & Associates, Attorney at Law;County Square Professional Offices, Suite 101, 674 County Square Drive, Ventura, CA, 93003, US

(21) Appl. No.: 11/417,968

(22) Filed: May 3, 2006

Related U.S. Application Data
(60) Provisional application No. 60/677,238, filed on May 3, 2005.

(51) Int. Cl. A61F 5/00

(52) U.S. Cl. 602/3; 602/18

(57) ABSTRACT

A cover for a cervical collar that is decorative, comfortable, washable and removable that eliminates the unsightly appearance of cervical collars, the problem of soiled cervical collars and the discomfort associated with the wearing of said cervical collars.

### PRIOR FILING

This application emanates from the prior filling of a Provisional Patent Application, Ser. No. 60/677,238, filed with the United States Patent & Trademark Office on 3 May 2006

### FIELD OF THE INVENTION

The invention disclosed herein relates to covers for prescribed medical aids and equipment. More particularly, the invention lies in the field of decorative covers for cervical collars.

### BACKGROUND OF THE INVENTION

After an injury to the neck, either through trauma, such as whiplash, or surgery, or vertebrae and disc degeneration typically encountered in certain vocations and old age declining years, a patient is often required to wear a cervical collar for a period of weeks, months, or even years. Cervical collars are typically made of a white or off-white color, are rough and stiff around the neck of the wearer and can be uncomfortable at the interface between the edges of the collar and the body of the wearer.

Never the less, wearing of cervical collars is a necessary step for proper healing of neck injuries, even though the wearing of the cervical collar can cause discomforts to the wearer. Cervical collars are typically unsightly, draw undesired attention to a patient's neck, and are embarrassing as the collar stands apart from anything else worn by the person wearing the cervical collar. In addition to being bulky and uncomfortable, cervical collars are easily soiled, can not be washed, and therefore tend to acquire a grungy look from normal accumulation of perspiration, body oils, makeup, and other particulate matter normally picked up from a normal environment.

Prior art in the field has dealt with some of the problems associated with the wearing of cervical collars, but the problem of unsightliness has heretofore been left unaddressed. U.S. Pat. No. 5,722,939 to Hohlen describes a body support liner for a neck brace. While the Hohlen invention involves a removable liner that is the shape of a cervical collar, the Hohlen invention does not expressly seek to solve, nor does it indeed succeed in solving the problems associated with the wearing of cervical collars other than the prevention of irritation associated therewith. The Hohlen collar invention is not expressly washable, hence it does not address the hygiene issue associated with cervical collars. Further, it does not solve the problem of the unsightliness and grungy look of cervical collars in general.

Other prior art has addressed the need for a removable, washable cover for cervical collars, but none have addressed the unsightliness associated therewith. These prior art include U.S. Pat. No. 6,926,686 B2 to Cheatham end U.S. Pat. No. RE 35,290 to Druskozel. Additionally, Maynard has two design patent, DES 422,710 and DES 355,400 for cervical collars, the “ornamental appearance” of which is significantly different from the tubular, cylindrical image described in the instant invention, and, of course, being a Design Patent, said art does not address, delineate, or claim the multipurpose function and intended purpose of the invention disclosed herein.

Similar limitation exist in yet another Design Patent of Follman, U.S. Pat. No. Des 519,639, which discloses a cervical collar cover somewhat in the nature of sock, with an apparent lack of “ornamental appearance” other than its structure, into which a cervical collar may be inserted.

### SUMMARY OF THE INVENTION

According to the present invention a cervical collar cover is disclosed that is approximately 3-5″ high and 10-15″ long, depending on the size of the cervical collar upon which the invention collar cover worn. For people wearing cervical collar after a neck injury, such as whiplash, old age, or recovery from surgery, the instant invention offers a clean, comfortable and decorative alternative to the ugly white bulky cervical collar worn alone.

The collar cover size varies according to the size of cervical collar worn, including small, medium, large and children's. The cover is made of a soft material, such as a knit, that is removable and washable. The thickness of the cover can be modified for increased comfort. The cover is made of a variety of colors and designs that complement the clothing worn by the wearer. The cover is tubular in shape and is easily slipped on over any size cervical collar worn.

The collar cover can be made in a variety of collar colors, fabric, or fabric designs. In addition to colors and patterns, decorative touches, such as rhinestones, ribbons, bows and the like, can added for decorative touch. During seasonal occasions, such as holidays, the covers can be made to include seasonal designs corresponding to the occasion. The cover can be pulled or slipped over the cervical collar for ease of application as well as removal. The collar cover is held fast in a non slip position by elastic elements on either end of the collar cover. It is further considered that the invention delineated herein allows for adaptation to partial cervical collars for adaptation to unique uniforms or attire that would not permit a full cervical collar.

### OBJECTS OF THE INVENTION

The principal object of the invention is to provide a cover for a cervical collar that is attractive, decorative, fashionable, and changeable.

It is yet another object of the invention to provide a cover for a cervical collar that is removable for washing.

It Is yet another object of the invention to provide a cover for a cervical collar that makes the wearing of the cervical collar more comfortable.

Numerous other advantages and features of and various means for practicing the invention will become apparent from the following detailed description of a preferred embodiment of the invention, from the claims, and from the accompanying drawing. In which like numerals are used to designate like parts shown in different figures.

### BRIEF DESCRIPTION OF THE DRAWINGS

The novel features of this invention will be best understood from the accompanying Figures, taken in conjunction with the accompanying description, in which similar characters refer to similar parts, and in which:

FIG. 1 is a perspective view of the cervical collar cover,

FIG. 2 is an end, elevational view of the cervical collar cover,

FIG. 3 is a perspective view of the cover applied to a cervical collar.

### DETAILED DESCRIPTION OF AN ENABLING AND PREFERRED EMBODIMENT

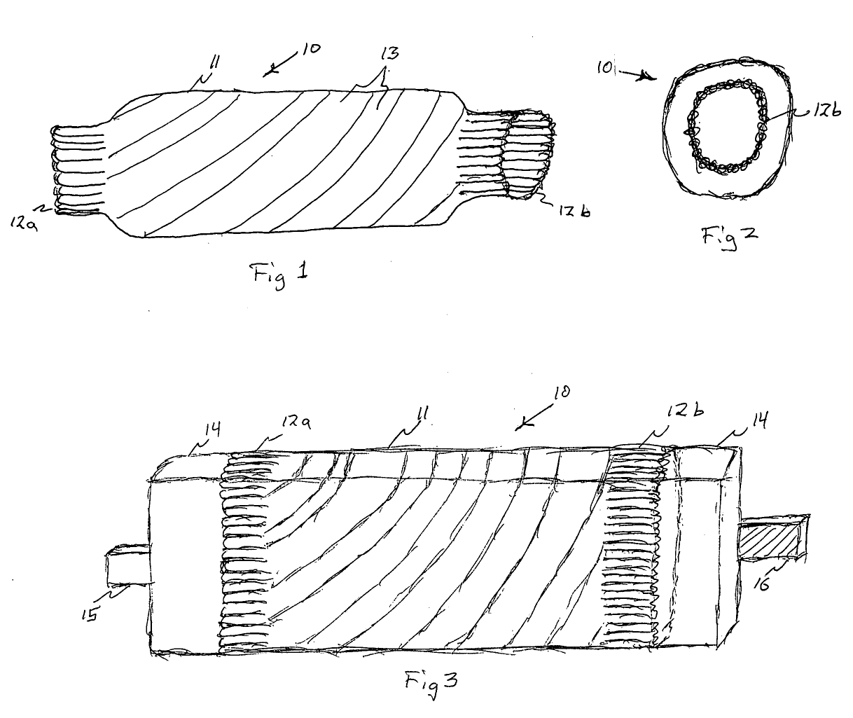

Referring now to the drawing, the collar cover 10, illustrated in perspective view FIG. 1, depicts a cylindrically shaped fabric or material defining its boundaries. FIG. 2 additionally illustrates an elevational end view of the cervical collar cover 10. FIG. 1 shows the cover 10 as it would appear prior application to a cervical collar. FIG. 3 illustrates cover 10 as it would appear mounted on a cervical collar 14 without fully covering the collar 14. The material of collar cover 10 is conceived to be of a type that can be streatched to cover only a portion of cervical collar 14 as illustrated in FIG. 3 or that may be streatched further to span the entire length, perimeter of collar 14.

Referring to FIGS. 1 and 3, cover 10 is composed of a main body portion 11 and two open end gripping portions 12a and 12b that allow for the cover 10 to slip over a cervical collar 14 and snuggly secure itself in a desired position thereon. Cervical collar 14 typically has a two part attachment means with a hook and loop assembly containing a hooking side 15 and a loop attachment side 16. The main body portion 11 of collar cover 10 and optionally the open end gripping portions 12a and 12b are conceived to have a decorative theme or color scheme 13 imprinted thereon.

While the particular invention, as herein shown and disclosed in detail, is fully capable of obtaining the objects and providing the advantages above stated, it is to be understood that the presently preferred embodiments are merely illustrative of the invention and no limitations are intended therefor.

I claim:

1. A decorative cover for use with a cervical collar comprising:
a cylindrically shaped main body;
first and second open ends on each end of said main body;
means disposed on each said open end for gripping and securing said cover to said collar; and
decorative means for displaying an image, design, and color on said cover.

2. A decorative cover according to claim 1, wherein said cover is made of a knitted fabric of varying degrees of thickness for varying levels of comfort.

3. A decorative cover according to claim 1, wherein said cover is made in a variety of sizes, lengths, and diameters designed to fit a variety of sizes of cervical collars.

4. A decorative cover according to claim 1, wherein said decorative means is designed to complement a clothing color worn by a person wearing said collar.

5. A decorative cover according to claim 1, wherein said decorative means is designed to match a clothing design worn by a person wearing said collar.

6. A decorative cover according to claim 1, wherein said decorative means is designed to celebrate a seasonal holiday.

7. A decorative cover according to claim 1, wherein said decorative means is designed for a festive occasion through the use of rhinestones, patchwork, ribbons, bows and the like.

8. A decorative cover according to claim 1, wherein said cover is made of a washable material.
